(19) United States
(12) Patent Application Publication
(10) Pub. No.: US 2026/0069761 A1
(43) Pub. Date: Mar. 12, 2026

(54) LIPOSUCTION AND FAT TRANSFER SYSTEM AND METHOD

(71) Applicant: Squlpt Management, LLC, Aventura, FL (US)

(72) Inventor: Daniel Suissa, Aventura, FL (US)

(21) Appl. No.: 19/327,315

(22) Filed: Sep. 12, 2025

Related U.S. Application Data
(60) Provisional application No. 63/693,862, filed on Sep. 12, 2024.

(51) Int. Cl. A61M 1/00

(52) U.S. Cl. CPC A61M 1/89

(57) ABSTRACT

An improved system and method for performing liposuction and autologous fat transfer is provided. The present invention comprises an improved lipoaspirate collection canister and multiple devices wirelessly and operably connected to one another, including a digital regulator box and wireless triple pedal unit for control of the system components. A liposuction handpiece is operably connected to the system devices, and a custom quad tubing is operably connected from the handpiece to the components of the system and supplies and/or removes fluids, fat tissue, compressed air, suction, and/or power for the procedure. The method disclosed herein includes using vibration to deliver tumescent fluid to a patient as well as to break up and remove fat tissue from a patient. During the fat preparation step, a vibrational plate is used to effectively and efficiently separate the lipoaspirate prior to transferring the fat tissue into the patient.

### CROSS-REFERENCE TO RELATED APPLICATIONS

This application claims priority to U.S. Provisional Patent Application No. 63/693,862, filed on Sep. 12, 2024, which is incorporated in its entirety herein by reference.

### TECHNICAL FIELD

The present invention relates generally to a device system and method of liposuction and fat transfer. More specifically, the present invention relates to a device system and method of liposuction and the separation of fat tissue from tumescent fluid and the preparation and transfer of fat tissue to a patient.

### BACKGROUND

In aesthetic body contouring, it is sometimes desirable to transfer fat tissue from one portion of the body where it is excessive or unwanted to another portion of the body where that same fat may be considered more appropriate or appealing. For instance, fat may be removed from a patient's abdominal, hip, or thigh region; it may be desirable to transfer this fat to the buttocks, breast, or bicep region, depending upon the patient. The transfer of fat from one region of a patient's body to another is commonly termed Autologous Fat Transfer or AFT.

The most common approach to fat removal is liposuction, which first involves the infiltration of fluid into a selected region of the body. The infiltrated fluid, comprising primarily a saline solution with an anesthetic (typically lidocaine) and a vasoconstrictor (typically epinephrine), serves several purposes. The bulk saline solution serves to expand the fat tissue away from the connective matrix and provide a more conducive fluid condition for fat removal, the lidocaine reduces pain and patient discomfort (even when the patient is fully sedated and unconscious for the procedure), and the epinephrine constricts the blood vessels, thereby reducing bleeding and extending the anesthetic effect of lidocaine. Once the infiltration fluid has been introduced, the fat/fluid mixture is removed using a vacuum or suction cannula. The cannula may also be mechanically actuated to assist in the removal.

The amount of fluid infiltrated into the patient is typically equal to, or more than, the expected amount of fat to be withdrawn. When the fat is removed by liposuction, it is in a lipoaspirate that includes remnant tumescent fluid, blood, and other unwanted connective tissues. In this form, it is neither optimal nor desirable for reinjection into the patient. Therefore, it is important that a means be available by which the wanted fat component can be separated from the overall lipoaspirate fluid before the fat component is reinjected into the patient.

To the maximum extent possible, it is also preferable that the process of harvest, separation, and reinjection of fat take place under conditions approaching aseptic. Doing so prevents the introduction of unwanted infectious or otherwise harmful material into the patient. While harvesting procedures have been well established and injection methods have also been well-described, there is nonetheless room for improved devices and techniques for these processes. Furthermore, separation techniques have received less focus and represent an underdeveloped area that is opportune for the overall improvement of the AFT process.

A number of approaches have been attempted to solve the separation process issues. In the simplest approach, the lipoaspirate is allowed to sit in a harvest container for a period of time as gravity takes effect and the innate buoyancy of the components within the solution causes natural separation. The fat component, being less dense than either blood, plasma, oil, or saline, eventually floats to the top region of the container where it can be decanted. This process is often referred to as the gravity method. However, the gravity method requires waiting a moderately long amount of time, and the separation is rarely complete in that much of the fat remains in mixture with the other components of the solution for a duration of time longer than the patient should ideally remain under anesthesia.

Another method involves pouring the lipoaspirate into smaller centrifuge tubes and centrifuging the fat for a period of time. This process more thoroughly separates the fat cells from the other fluids, and also significantly reduces the amount of time required to complete the separation. The centrifuge tubes can either be decanted into syringes, or, in some systems, capped syringe tubes are used within the centrifuge, eliminating one step in the process.

Another method involves pouring the lipoaspirate through a metal sieve or colander, capturing the fat above the interface and allowing the solution to be collected for discard. The fat is then manually scooped into syringe tubes. This is referred to as the colander method.

Yet another method involves pouring the lipoaspirate onto a sterile absorbent pad and manipulating the fat on the pad until the desired amount of fluid has been absorbed and the remaining fat is sufficiently dry. This technique has been named “Telfa rolling.”

The above techniques involve exposing the lipoaspirate to the open environment, with manual handling and manipulation. Thus, they all suffer from an increased possibility of microbial contamination. Furthermore, these methods are generally suited to batch operation, which is less efficient than continuous operation, and typically involve only small quantities of fat that do not answer the needs of various use cases of AFT.

Alternative “closed” approaches exist such as using a container with a removal port at the bottom of the vessel, which eliminates the need to decant the fat. Instead, after the necessary settling time, the bottom valve is opened and the (heavier) blood/fluid is drawn off until only the fat remains. The port is then connected to an injection syringe and the fat is transferred for reinjection. The nomenclature for this is traditionally the evacuation port technique.

Another approach involves placing a filter within a lipoaspirate container, such that the fat is captured and the unwanted liquids are conveyed to another disposal container.

Another approach involves pumping the lipoaspirate into a flexible, disposable plastic container with an internal filter. The fat is further processed by the introduction of additional saline solution, with manual agitation of the container. The result of this process is a relatively pure fat cell solution. However, the amount of fat processed is limited to the size of the flexible container, which is less than 500 cc. The design also limits the process to a small batch at a time. Lastly, this method does not handle fat that is removed by technologies such as power-assisted lipoplasty (PAL) very well because of the large strands of fat and connective tissue that are encountered that cause clogs and blockages in the apparatus. This method is referred to as pouch preparation.

The prior techniques and devices have various shortcomings, including: 1) long processing times 2) limited total fat processing capability 3) utilization of an open system that exposes the fat to the external environment and increases the risk of microbial contamination; 4) small volume “batch” processing whereby individual syringe tubes are filled with fat for manual injection, a method that is inefficient for higher fat volumes, allows for erroneous over-bolus of fat that may lead to fat necrosis and oil cysts, provides the surgeon less control over the location of the cannula and its tip, and confers a higher risk of accidental intravascular injection.

Given the limitations and problems associated with the systems discussed above, it would be advantageous to provide a novel liposuction and fat transfer system and process which allows for a procedure to be performed more quickly and efficiently than conventional methods, which more effectively prevents microbial contamination and infection, and which reduces the risk of intravascular injection and its potentially catastrophic consequences.

### SUMMARY

The present invention generally relates to a system and method of liposuction and autologous fat transfer, further including preparing tissue extracted by liposuction for transplantation. More specifically, the present invention relates to a system and method of separating extracted fat tissue from tumescent fluid prior to transferring the prepared fat tissue to another area of the body.

Embodiments of the present invention include a digital air pressure regulator with custom printed circuit board, quad tubing for ease and simplicity of connecting the components of the system, a triple control pedal with wireless connection for wireless control of the system, and a fat collection canister and vibrational plate for aseptic and efficient separation of lipoaspirate in preparation for the fat transfer process.

According to one or more embodiments of the present invention, the liposuction and fat transfer system of the present invention is comprised of multiple devices operably connected to provide an improved system and method for performing liposuction and autologous fat transfer. System devices of the present invention may include a fully digital and computerized regulator box for controlling the air pressure and other equipment as discussed herein, a peristaltic pump operably and wirelessly connected to the digital regulator box, a suction machine operably and wirelessly connected to the digital regulator box, a fat collection canister operably connected to the suction machine and the peristaltic pump, a vibrational plate for preparing the fat tissue for transfer into the patient, and a liposuction handpiece operably and simultaneously connected to the regulator box, the peristaltic pump, the suction machine, and the fat collection canister. A triple pedal system includes a wireless transmitter for wireless control of the devices listed above, and a quad tubing is operably connected from the handpiece to the devices above and is utilized to supply and/or remove fluids and fat tissue, compressed air, suction, and/or power for the procedure.

The liposuction and fat transfer method disclosed herein includes using vibration throughout various steps of the procedure. During the liposuction step, vibration is used to deliver tumescent fluid to a patient as well as to break up and remove fat tissue from undesired locations in a patient's body. During the fat preparation step, vibration is used to effectively and efficiently separate the suctioned fat tissue from tumescent fluid and wash fluids prior to transferring the fat tissue into the patient.

### BRIEF DESCRIPTION OF THE DRAWINGS

The foregoing, as well as the following Detailed Description, is better understood when read in conjunction with the appended drawings. For the purposes of illustration, there is shown in the drawings exemplary embodiments; however, the presently disclosed subject matter is not limited to the specific methods and instrumentalities disclosed.

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1 is a perspective view of an embodiment of the system set-up including the peristaltic pump, the digital regulator box, the suction machine, and the fat collection and transfer canister.

FIG. 2 is a perspective view of an embodiment of the triple pedal with wireless receiver.

FIG. 3 is a front view of an embodiment of the digital regulator box and operably connected liposuction handpiece.

FIG. 4 is a rear view of an embodiment of the regulator box.

FIG. 5. is a perspective view of an embodiment of air compressor attachments on the side of the regulator box.

FIG. 6 is a perspective view of an embodiment of the regulator box showing the attachments on the lateral side of the box.

FIG. 7 is a perspective view of an embodiment of the quad bonded tubing.

FIG. 8 is a front view of an embodiment of the peristaltic pump.

FIG. 9 is a perspective view of an embodiment of the fat collection canister.

FIG. 10 is a perspective view of an embodiment of the fat collection canister and the vibrational plate.

FIG. 11 is a perspective view of an embodiment of the fat collection canister attached to the vibrational plate.

FIG. 12 is a cross-sectional view of an embodiment of the fat collection canister illustrating one embodiment of the hollow interior pathway of the elbow fitting.

FIG. 13 is a perspective view of an embodiment of the elbow fitting of the fat collection canister.

FIG. 14 is a perspective view of an embodiment of the canister after fat has been collected, further illustrating the short connector tube and clamp.

FIG. 15 is a perspective view of an embodiment of the fat collection canister after placement on the vibrational plate.

FIG. 16A is a perspective view of collected fat in the canister, prior to separation.

FIG. 16B is a perspective view of separated fat in the canister after fat has been collected and separated, but prior to being transferred into the patient.

FIG. 17 is a flow chart illustrating signal processing in the wireless regulator box.

FIG. 18A illustrates an embodiment of the fat transfer tubing as it connects with the fat transfer cannula.

FIG. 18B illustrates an embodiment of the intermediate threaded fitting encompassing inner blades to further cut fat tissue prior to entering the fat transfer cannula.

FIG. 19 is a perspective view of an embodiment of the fat collection canister mounted on the vibrational plate.

FIG. 20 is an exploded view of an embodiment of the fat collection canister.

FIG. 21 is a front view of an embodiment of the fat collection canister mounted on the vibrational plate.

FIG. 22 is a side view of an embodiment of the fat collection canister mounted on the vibrational plate.

FIG. 23 is a cross-sectional side view of an embodiment of the fat collection canister mounted on the vibrational plate.

FIG. 24 is a perspective view of an embodiment of the fat collection canister, according to one or more embodiments.

FIG. 25 is a front view of an embodiment of the fat collection canister.

FIG. 26 is a side view of an embodiment of the fat collection canister.

FIG. 27 is a cross-sectional side view of the fat collection canister, illustrating an embodiment of the aeration splitter and elbow fitting.

FIG. 28 is a perspective view of an embodiment of the aeration splitter.

FIG. 29 is a top view of an embodiment of the aeration splitter.

FIG. 30 is a side view of an embodiment of the aeration splitter.

FIG. 31 is a cross-sectional top view of an embodiment of the aeration splitter.

FIG. 32 is a cross-sectional front view of an embodiment of the aeration splitter.

### DETAILED DESCRIPTION

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that same thing can be said in more than one way.

Alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure.

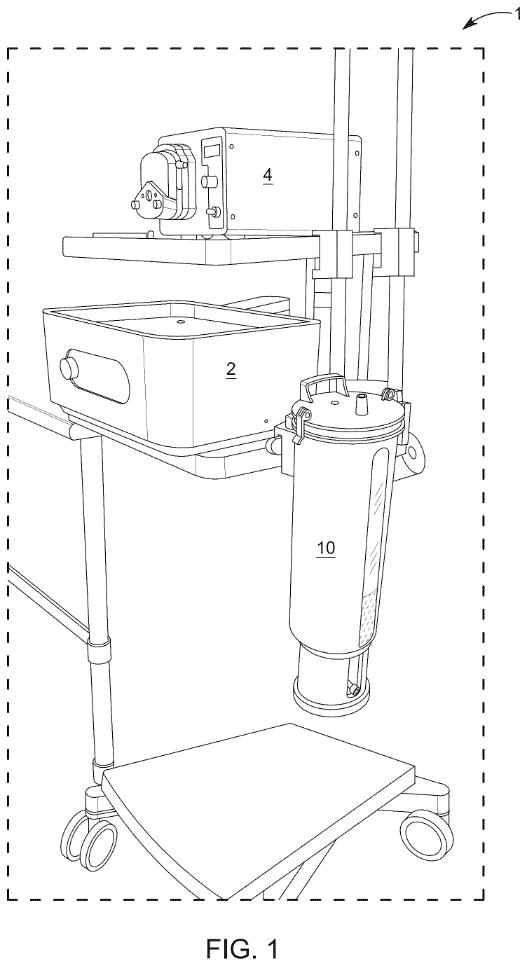

FIG. 1 illustrates an embodiment of the liposuction system 1 setup comprising a digital regulator box 2, a peristaltic pump 4, and a suction machine 6. In the configuration depicted in FIG. 1, a fat collection canister 10 is operably coupled to the suction machine 6 and/or the peristaltic pump 4. Conventionally, systems for liposuction procedures comprise a plurality of wires spanning the operating suite, often routed along the floor near the surgeon and medical staff. Such wiring presents safety hazards and increases the risk of compromising operating room sterility.

The system 1 of the present invention addresses these deficiencies by implementing a wireless configuration. Wireless transmitters and receivers, including but not limited to Bluetooth modules, are operatively coupled to the peristaltic pump 4, air pressure regulator 2, suction machine 6, and triple control pedal 14, thereby permitting wireless operation. This arrangement simplifies system setup and reduces the total amount of physical tubing and wiring required for device interconnection and operation.

The liposuction handpiece 8 disclosed herein is fully described in U.S. Pat. No. 12,090,263 and is incorporated herein by reference. The handpiece 8 comprises a distal end configured for selective attachment of various cannulas, and a proximal end defining four respective ports: a compressed air-in port, a compressed air-out port, an infiltration fluid delivery port, and a suction port. In use, the handpiece 8 may be simultaneously operably coupled to system 1 devices, such that individual disconnection and reconnection of lines, wires, or tubes for different procedural steps is unnecessary. System 1 devices include, but are not limited to, a regulator box 2 for controlling flow of compressed air into the system 1 which powers the handpiece 8 engine and generates vibration; a peristaltic pump 4 for tumescent solution delivery; and, a suction apparatus 6 for removal of fat.

Furthermore, the system 1 comprises a fat transfer cannula 98 operably connectable to the fat collection canister 10 via the peristaltic pump 4, enabling closed-system delivery of collected fat 88 from the canister 10 to the cannula 98. In this manner, the fat transfer process can be precisely controlled by toggling the foot pedal 14 of the peristaltic pump 4, alternatively initiating or stopping the flow of fat from the canister 10 to the fat transfer cannula 98, thereby obviating the need for filling a plurality of individual syringes for reinjection. This closed-system fat transfer process is accomplished by a fat transfer tube 96 extending from the collection canister 10 through the peristaltic pump 4, which advances the fat at a controlled rate from the canister 10 directly to the cannula 98.

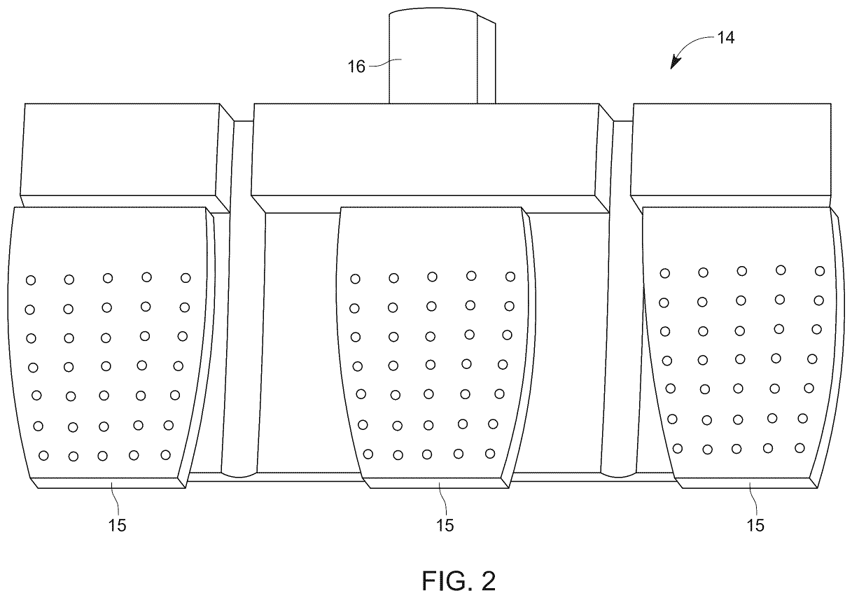

The present system 1 includes devices that may be selectively controlled or actuated by means of a pedal unit 14, wherein said pedal unit 14 may be placed in any position convenient for the operator. In a preferred embodiment, the pedal unit 14 is a wireless triple pedal unit comprising a wireless transmitter 16 operatively coupled thereto, and is configured to transmit activation signals to corresponding wireless receivers operably associated with respective devices of the system 1. FIG. 2 depicts an embodiment of such a triple pedal 14 provided with an integrated wireless transmitter 16.

The triple pedal unit 14 is constructed as a single module comprising three discrete pedals 15 on one convenient platform, each pedal 15 being configured to independently activate or control a separate device. By way of non-limiting example, a first pedal may be configured for selective activation and deactivation of the air compressor regulator 2, a second pedal for selective activation and deactivation of the tumescent fluid peristaltic pump 4, and a third pedal for selective activation and deactivation of the suction machine 6. Upon depression of a respective pedal 15, the wireless transmitter 16 of the triple pedal unit 14 communicates an activation signal to the wireless receiver of the corresponding device associated with the actuated pedal 15.

A plurality of wireless triple pedal units 14 may be simultaneously employed with the system 1, thereby permitting triple pedal unit 14 placement at multiple operative locations, such as on one or both sides of the patient's surgical bed, or at any other location as required during the surgical procedure, thereby enhancing system flexibility and ergonomic convenience for surgical staff.

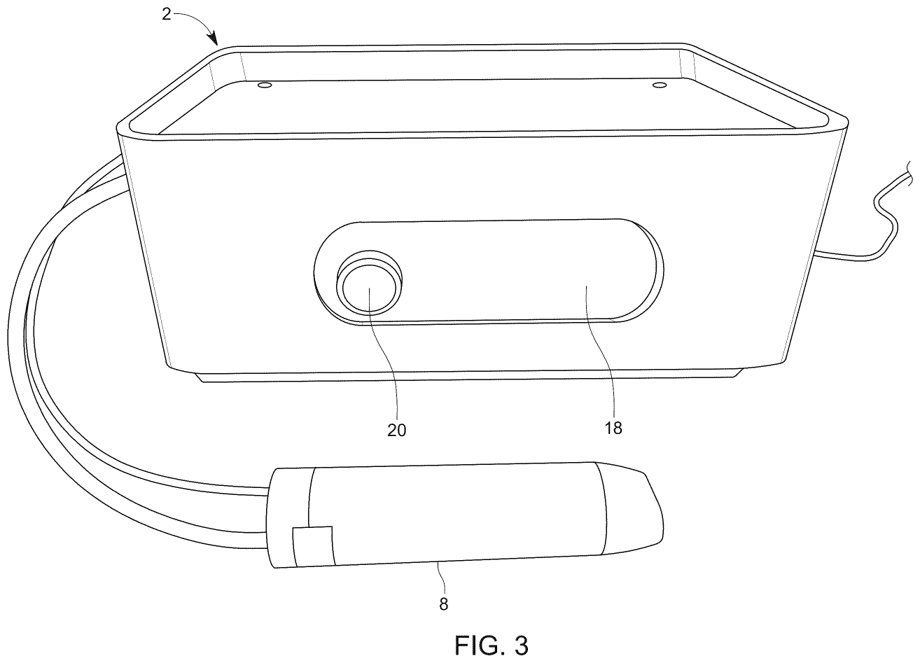

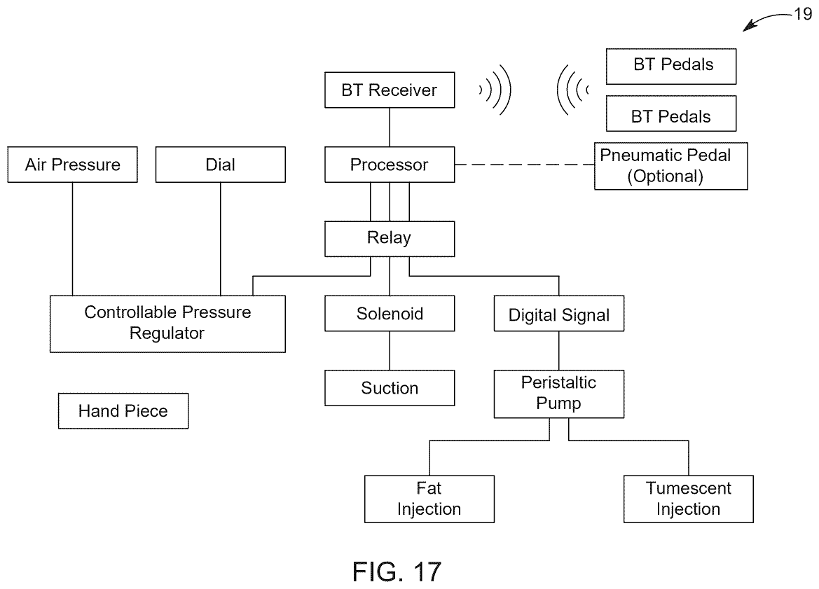

The digital regulator box 2 (also referred to herein as “regulator box”, “air pressure regulator”, and “air compressor regulator”), as illustrated in FIGS. 3 through 6, is an electronically controllable air pressure regulator 2 enabled with wireless communication capabilities, designed for use in liposuction procedures. The regulator box 2 incorporates a custom printed circuit board (PCB) in a central processing unit 19 that can initiate processes, depicted in FIG. 17. This central processing unit 19 serves as a central command module for the entire integrated liposuction and fat transfer system 1.

The digital regulator box 2 further comprises the capability to simultaneously activate suction 6 and the peristaltic pump 4 via the triple pedal unit 14. The air pressure regulator 2 supports operation by both wireless and/or Bluetooth-enabled pedals and pneumatic pedals, each configured to control the airflow to the air pressure-powered liposuction handpiece 8. The air pressure regulator 2 allows the user to primarily deploy wireless pedals 15 to control the delivery of compressed air to the liposuction handpiece 8. The compressed air powers an internal engine which generates vibration within the handpiece 8. In the event that the wireless pedals 15 malfunction, the regulator box 2 will allow backup pneumatic pedals to be connected for control of compressed air. Activation signals from either pedal type are transmitted to the central processing unit 19, which in turn actuates a multi-channel relay to power each respective device independently.

As shown in FIG. 3, a user interface 18 located on the front panel of the regulator box 2 includes a rotary dial 20 to enable precise user control over the air pressure delivered to the handpiece 8. An electronic display mounted on the front thereof provides a real-time indication of the air pressure at the handpiece 8, accurately, preferably in increments of one pound per square inch (1 PSI).

The wireless pedals unit 15 manages the activation and deactivation of suction through a solenoid valve and control the peristaltic fluid pump 4 using digital signaling. Both the suction solenoid and the peristaltic pump 4 are physically external to the regulator box 2 and connect thereto via rear-panel cabling.

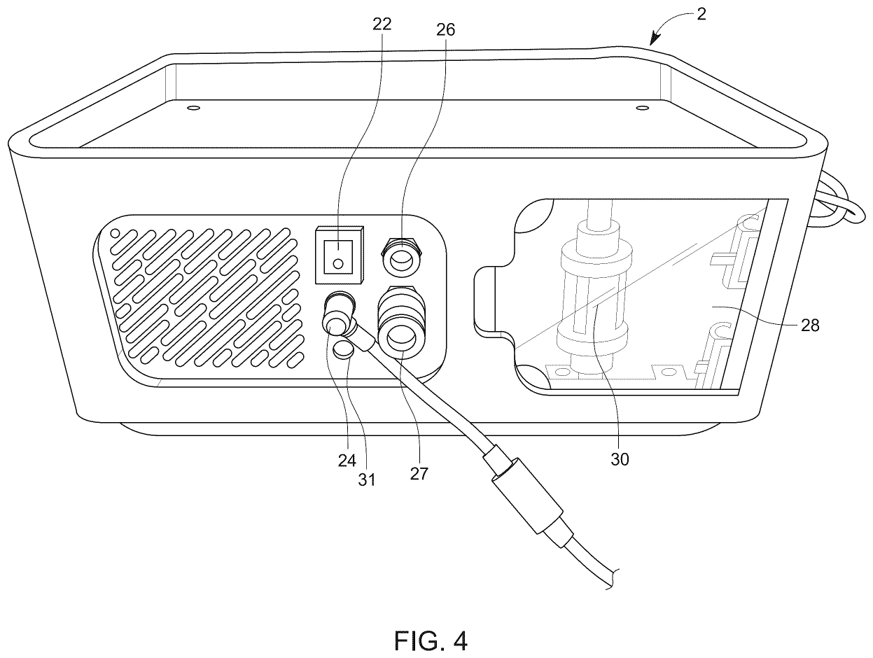

FIG. 4 illustrates an embodiment of the rear portion of the digital regulator box 2. The regulator box 2 includes a power switch 22 operably coupled to a power source, enabling electrical activation and deactivation of the regulator box 2. Adjacent to the power switch 22 is a pneumatic fitting 24 for connecting the backup pneumatic pedals in tandem to the regulator box 2. Adjacent this pneumatic fitting 24 are connectors 26, 27 for attaching the compressed air line to the regulator box 2. These fluidic connectors are designated as compressed “air-in” 27 and compressed “air-out” 26 ports.

FIG. 4 also shows an embodiment of a transparent door panel 28. Compressed air returned from the liposuction handpiece 8 passes through the regulator box 2 via the “air-in” connection 27, carrying entrained contaminants including lubricating oil. Such contaminants are captured by an oil filter 30 integrated into the regulator box 2. Unlike regulator boxes where the oil filter is concealed internally, this design provides for external visibility and accessibility of the oil filter 30 to facilitate timely maintenance, thereby preventing oil leakage into the compressor 2 machinery.

Both the wireless receiver 31, the oil filter 30, and a muffler are accessible externally at the rear of the regulator box 2, eliminating the need for disassembly during routine maintenance, thus yielding a more compact and organized system configuration with reduced tubing and wiring complexity within the operative environment. In this way, a user can monitor the oil filter 30, the muffler, and other internal electronics, and determine more precisely when these components need to be changed, thereby simplifying maintenance of the device. In a preferred embodiment, the oil filter 30 is observable through a transparent panel 28 covering the filter assembly to enable visual inspection and timely replacement.

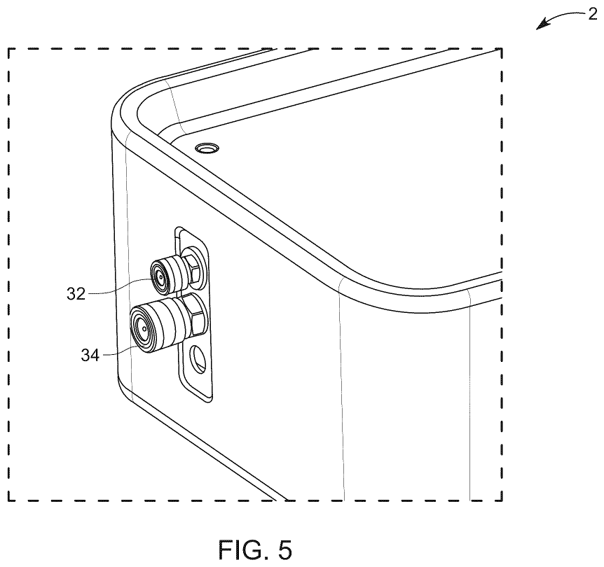

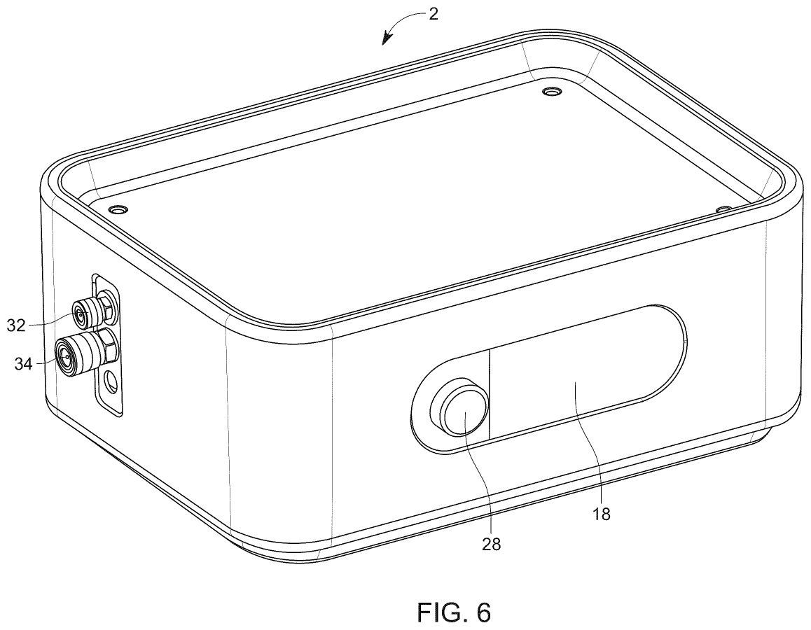

FIGS. 5 and 6 depict lateral views of the regulator box 2, highlighting additional compressed “in port” 32 and “out port” 34 connection ports. The “in port” fitting 32 is adapted to direct a flow of compressed air from the regulator box 2 into the handpiece 8 via an air-in tubing 44, the air-in tubing 44 constituting one of the four tubular pathways of the bonded tubing 36 assembly discussed further herein. Within the handpiece 8, the compressed air is operatively introduced into an internal pneumatic engine configured to generate vibrational energy, which is subsequently transmitted through an associated cannula. The vibrational energy is selectively actuated by a user via the foot pedal 14 operatively coupled to the regulator box 2.

When the foot pedal 14 is toggled to the “on” state, an internal control valve of the regulator box 2 is actuated to an open position, thereby permitting compressed air to be discharged from the regulator box 2 into the air-in tubing 44 for delivery to the handpiece 8. Upon circulation through the handpiece 8, the compressed air is exhausted via an air-out tubing 46. The air-out tubing 46 directs the exhaust flow back toward the regulator box 2 and interfaces with the “out port” fitting 34. The out port 34 is configured to receive the exhaust flow of compressed air and route the received flow into the regulator box 2 for subsequent treatment, including (i) passage through an oil filter 30 to remove lubricant constituents as previously disclosed, and (ii) passage through an acoustic muffler to attenuate operational noise, thereby enhancing the comfort and overall experience of both the user and the patient. The influx of compressed air actuates vibration within the handpiece 8, the intensity and frequency of which are directly proportional to the air pressure delivered.

The degree of vibration is regulated via the rotatable dial 28 located on the regulator's front panel, which correspondingly adjusts the air pressure in increments displayed digitally from 0 to 100, each increment representing approximately 1 PSI. Higher displayed values correlate to increased vibration amplitude (“intensity”) and frequency (“speed”).

The custom printed circuit board incorporated within the regulator 2 facilitates precise and accurate modulation of air pressure, thereby allowing refined control and titration of handpiece 8 vibration during fluid infiltration and liposuction procedural steps. Typical operating parameters involve air pressures on the order of 70 to 80 PSI, with an optimal setting of approximately 75 PSI, although variations above or below these values are possible according to clinical requirements, such as to match the comfort level of the patient, or to accommodate for removing fat from areas of the body invested with dense connective tissue, or to facilitate revisional procedures that work through tough, contracted areas of scar tissue.

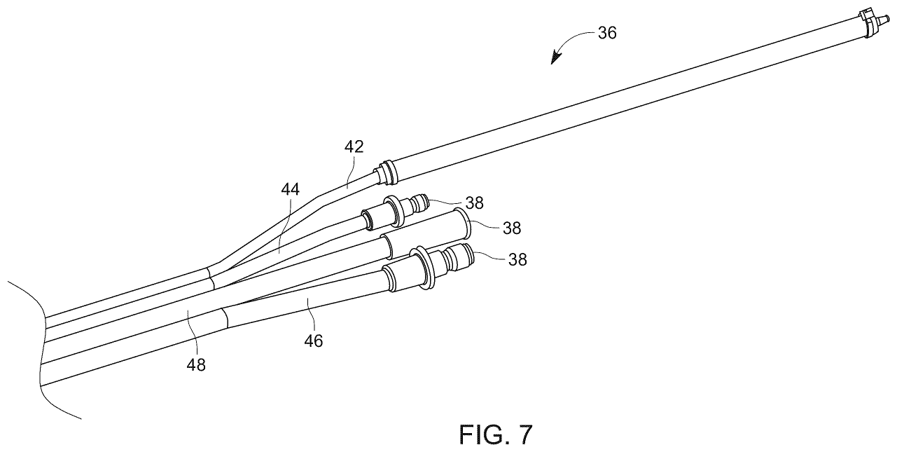

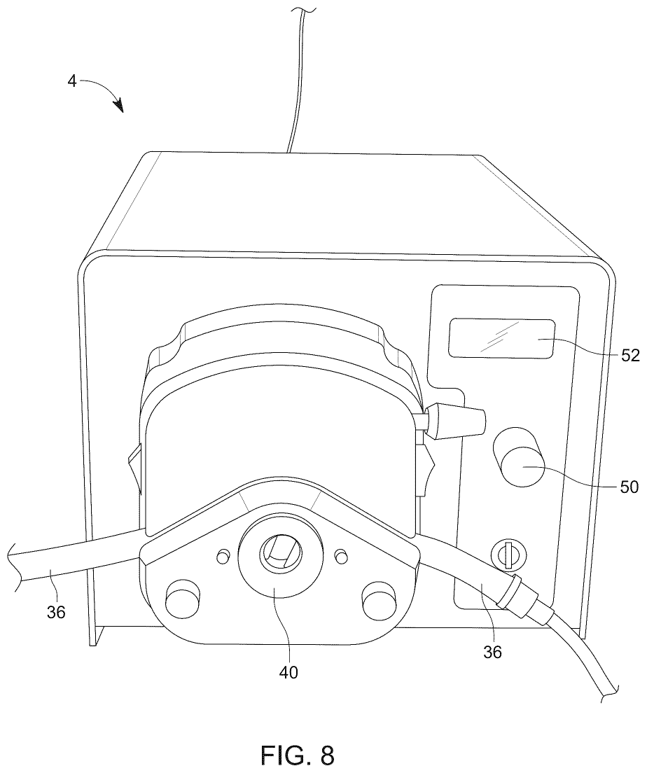

The present system 1 also utilizes customized, disposable surgical grade tubing assembly 36, illustrated by FIG. 7. This tubing assembly 36 may be parallel tubing (or paratubing) and is customized for this system 1. With paratubes, the tubes are produced as multiple single lumen PVC tubes that are longitudinally bonded together in a set of more than one tube (in this case a set of at least four) and can be peeled apart when and where needed along the paratube. Alternatively, the tubing assembly 36 may be designed separately, or only connected in parallel between two tubes or three tubes. In one or more embodiments of the present system 1, a single, disposable pack of quadruple-bonded tubing 36 includes four separable tubes having proximal and distal ends. The proximal ends can be simultaneously operably connected to the regulator box 2, the peristaltic pump 4, the tumescent solution bag, and the fat collection canister 10. The distal ends can be simultaneously operably coupled to the four corresponding functional connection points at the rear of the handpiece 8. For clarity, these four tubes will be designated herein as 1) air-in tubing 44; 2) air-out tubing 46; 3) tumescent fluid tubing 42; and, 4) suction tubing 48. In a preferred embodiment, the tubes have an inner diameter of 5 mm and are compatible to fit the functional attachment points of the handpiece 8 and the other system 1 components.

FIG. 7 also illustrates the custom-molded fittings 38 that are dimensioned based on compressed air fitting geometries, wherein each fitting is ultrasonically welded to the exterior surface of the tubing such that the inner diameter of both the fitting 38 and the underlying tubing remains substantially uniform throughout the connection interface. In contrast, conventional connection means utilize metal barbed end fittings that are inserted into the tubing, thereby reducing the inner diameter of the tubing at the point of insertion and impeding consistent airflow. Moreover, traditional metal fittings must be removed from the tubing for sterilization, whereas the custom-molded fittings 38 disclosed herein are fabricated from disposable materials, such as plastic, thereby obviating the need for post-use sterilization procedures. Additionally, conventional fittings often employ push-to-connect attachment mechanisms that are susceptible to separation from the air compressor 2 or tubing under high-pressure conditions. The custom fittings 38 of the present system 1 are specifically engineered to withstand increased pressure differentials without detachment, thereby providing a more secure and reliable pneumatic connection under operative conditions.

In preparation for the anesthesia step of the procedure, a sterile tumescent fluid is formulated by combining selected active ingredients within a sterile, saline-based intravenous (IV) bag. In a preferred embodiment, the tumescent solution comprises lidocaine, epinephrine, and a buffering agent admixed into a saline carrier medium. By way of non-limiting example, a representative formulation of the tumescent solution includes approximately 300 mg of lidocaine, approximately 1 mg of epinephrine, and approximately 12.5 mEq of sodium bicarbonate, the foregoing active agents being added to a 1 liter solution of 0.9% normal saline.

The inclusion of epinephrine in the formulation functions to induce localized vasoconstriction of capillaries, thereby reducing intraoperative bleeding and prolonging the duration of anesthesia. The lidocaine component functions as a local anesthetic agent, providing localized analgesia to the surrounding tissue structures. The buffering agent facilitates maintenance of physiologic pH in the solution, thereby enhancing patient tolerability and reducing injection-associated discomfort.

The tumescent tubing 42, which is preferably transparent to enable visual verification of fluid presence and flow, comprises at its proximal end a hollow-bore spike that is configured for insertion, consistent with standard sterile protocol, into an IV bag containing a pre-mixed tumescent solution. Following insertion, the tubing is routed around and operably secured within a roller mechanism 40 of the peristaltic pump 4. The peristaltic pump 4 comprises a drive motor which, in a preferred embodiment, is activated by the surgeon via the foot pedal 14. When the surgeon toggles the foot pedal 14 to the “on” state, a wireless signal is transmitted to a receiver 31 disposed within the regulator box 2. The receiver 31 communicates a control signal to the peristaltic pump 4 to initiate operation of the roller 40 drive motor.

When actuated, the roller mechanism 40 of the peristaltic pump 4 sequentially compresses and releases the tumescent tubing 42 to draw the tumescent solution from the IV bag and propel the solution toward the distal extent of the tubing 42. The flow rate of the solution is variably adjustable via a rotation knob 50, with a dynamic range accommodating a minimum flow rate of approximately 0.1 mL/min up to a maximum of approximately 1040 mL/min. The selected flow rate is indicated on a user-display interface 52 disposed on the frontal housing of the peristaltic pump 4.

At the distal end, the tumescent tubing 42 is fluidly coupled to the rear portion of the handpiece 8 by means of a barbed connector disposed at a tumescent port. From the tumescent port, the solution is conveyed into an internal fluid channel configured within the handpiece 8 body. The fluid channel is in direct fluid communication with a tumescent infiltration cannula. The infiltration cannula is provided with a plurality of fluid egress apertures disposed in proximity to its distal tip. The apertures form a delivery interface whereby the tumescent solution is dispensed directly into the patient's target tissue volumes.

In an alternative embodiment, the pump mechanism may include a syringe pump or diaphragm pump in place of the peristaltic pump, the activation of which may be similarly controlled via either wireless actuation, direct wired interface, or manual activation. In further embodiments, the flow-control mechanism may be substituted with electronic or digital control systems, wherein the surgeon or user may adjust rate of delivery via touchscreen, programmable presets, or automated delivery algorithms responsive to patient-specific inputs.

In another embodiment, the cannula design may vary in terms of length, bore diameter, or number and arrangement of apertures to accommodate different anatomical sites or desired infiltration patterns. For example, the apertures may be arranged circumferentially, linearly, or in staggered positions along the distal tip to provide uniform, diffuse, or directional delivery of solution. In still further embodiments, the cannula may be rigid, semi-rigid, or flexible, or may include a detachable or replaceable tip to optimize its infiltration characteristics.

In yet another embodiment, the tubing may further include in-line check valves, filters, or pressure monitoring sensors configured to increase delivery accuracy or provide feedback data to the operator. Such alternative arrangements may be employed independently or in combination with the embodiments described above, all falling within the intended scope of the present disclosure.

During the step of administering local anesthesia to a target body treatment area, the operating surgeon activates and coordinates operation of multiple components of the system 1 in sequence. Initially, the surgeon creates a small adit in the patient's skin providing an entry site for a tumescent infiltration cannula. Thereafter, the surgeon initiates the vibration mechanism of the handpiece 8 and its coupled cannula. In a preferred embodiment, this activation is accomplished via depression the foot pedal 15 disposed upon a wireless triple pedal 14 platform. The foot pedal 15 transmits a signal to the regulator box 2, thereby actuating a control valve configured to introduce compressed air into the handpiece 8. Upon activation, the handpiece 8 generates mechanical vibrational waves which propagate along the length of, and through, the attached tumescent cannula.

While the cannula is vibrationally actuated, the surgeon advances the cannula through the created adit into subcutaneous tissue of the treatment site. Once appropriate cannula entry is achieved, the surgeon selectively toggles a second foot pedal 15 control configured to activate the peristaltic pump 4 of the system. Upon activation, the peristaltic pump 4 withdraws pre-mixed tumescent solution from the IV bag, displaces said solution forward through the tumescent tubing 42, delivers the solution into an internal tumescent channel of the handpiece 8, and subsequently propels the solution through the tumescent cannula for infiltration into patient tissues.

The surgeon manipulates the vibrating cannula along variable trajectories comprising multiple angles, depths, and orientations relative to the patient's anatomy, thereby facilitating dispersal and layering of the tumescent solution within targeted soft tissues and adipose regions. Such controlled infiltration produces an optimized local anesthetic effect by distributing solution evenly across treatment zones.

The integration of vibration during infiltration yields multiple clinically advantageous effects. The application of vibrational input induces analgesic effect based upon the gate control theory of pain, which posits that targeted non-painful stimuli at key anatomical locations function to inhibit or diminish the transmission of nociceptive signaling. Consequently, activation of vibrational energy concurrently with injection of the tumescent solution decreases perceived patient discomfort and diminishes pain signal propagation.

Additionally, the propagation of vibrational waves along the cannula amplifies dispersive forces at the cannula tip, thereby enhancing infiltration dynamics. Specifically, vibrational motion augments the radial and diffuse spread of the tumescent solution within adipose tissue, producing a broadened field of distribution and facilitating more homogeneous penetration of local anesthetic solution.

In one alternative embodiment, the vibrational mechanism may be actuated by means other than pneumatic compressed air, including but not limited to piezoelectric, electromagnetic, or ultrasonic vibration generators operably integrated into the handpiece 8. In another embodiment, activation control may be combined into a single multifunction foot pedal or alternatively by a hand-operated switch disposed directly on the handpiece, thereby allowing direct manual actuation.

In further embodiments, the infiltration cannula may incorporate variable tip geometries or port arrangements to modulate solution dispersion. For example, multiple linear egress apertures, spiral orientations, or controlled micro-ports may be integrated to tailor the infiltration pattern according to surgeon preference or procedural requirements.

In yet another embodiment, automated control protocols may be employed whereby the regulator box 2 controls vibration intensity and pump flow rate according to pre-programmed profiles, patient biometric data, or surgeon-selected presets. The system 1 may include feedback sensors and real-time monitoring of solution flow and cannula resistance to dynamically adjust output parameters for increased procedural safety and efficacy.

Following infiltration of the tumescent solution into the selected treatment area, sufficient anesthesia of the target tissue is achieved, and the procedure progresses to the liposuction phase. In preparation, the tumescent infiltration cannula is disengaged from the handpiece 8, and in its place a suction cannula is operably inserted and secured.

The central suction source, such as a wall-mounted vacuum machine, is operably connected to the fat collection canister 10 by means of rigid silicone tubing. The foregoing suction connection tubing is distinct from and independent of the quadruple-bonded PVC tubing assembly described previously. The rigid silicone tubing transmits sub-atmospheric or negative pressure, generated by the suction source, to the fat collection canister 10 in a uniform and consistent manner.

The fat collection canister 10 is further placed in fluid communication with the proximal suction tubing 48 segment housed within the quadruple-bonded tubing assembly. The proximal suction tubing 48 is configured to engage a barbed port provided in the lid of the fat canister 10, while its distal end engages a barbed fitting disposed at the posterior aspect of the handpiece 8. In this arrangement, sub-atmospheric pressure generated in the fat collection canister 10 is operably transmitted through the suction tubing 48, through the handpiece 8, and through the lumen of the cannula, extending distally to the cannula working tip aperture(s). Accordingly, the negative pressure at the distal tip of the cannula produces a localized vacuum effect, which facilitates aspiration and transport of adipose tissue and associated fluids (i.e., lipoaspirate) into the fat collection canister 10.

Analogous to the method of using vibration during infiltration of tumescent solution, vibration may be simultaneously incorporated during liposuction. In a preferred embodiment, activation of handpiece 8 vibration is accomplished when the surgeon actuates the foot pedal 15 operably linked to the regulator box 2. Upon depression of the foot pedal 15, a wireless signal is received at the regulator box 2, which in turn actuates a valve allowing compressed air to circulate within the handpiece 8. Circulating compressed air powers an internal pneumatic engine, generating vibration that is transmitted along the suction cannula.

The surgeon subsequently guides the vibrating suction cannula through the preformed skin adits into the adipose tissue layers of the patient. Once introduced, the surgeon may then actuate a foot pedal 15 configured to control connection to the central suction machine. The pedal 15 transmits a signal to the regulator box 2, which communicates with the wall suction unit, causing a solenoid valve therein to open and establish sub-atmospheric pressure throughout the suction pathway. In this configuration, vacuum pressure is transmitted seamlessly from the central suction machine into the fat canister 10, through the connected tubing 48, through the handpiece, and ultimately to the suction cannula tip, wherein lipoaspirate is removed from the patient.

The system 1 herein provides the surgeon with multiple operative modalities, including suction with vibration, suction without vibration, and vibration without suction. In the suction-with-vibration mode, the system enables simultaneous mechanical vibration and lipoaspiration, thereby enhancing adipose disruption and facilitating more efficient fat removal. In the suction-without-vibration mode, the system permits conventional aspiration when vibrational assistance is unnecessary or undesired. In the vibration-without-suction mode, the system supports surgical techniques such as pre-tunneling and post-tunneling. Pre-tunneling involves the use of cannula vibration prior to activation of suction, thereby gently mobilizing adipose cells from their surrounding connective tissue matrix to reduce aspiration force requirements and improve tissue preservation. Post-tunneling involves the application of cannula vibration following completion of aspiration, wherein the residual adipose tissue beneath the skin is redistributed to achieve an improved and more uniform surface contour of the treatment area.

For the fat transfer step of the procedure, the fat must be separated from the tumescent fluid and any other fluids that have been suctioned from the patient prior to transferring the fat back into the desired body location. In conventional methods, a plastic container may be used to collect the lipoaspirate as it is suctioned from the patient. To separate the fat from the fluid, the container must sit for a period of time to allow for the fat tissue to rise to the top of the container where it can be decanted. This technique results in hypoxia of the fat tissue as it sits in the canister as well as prolonged anesthesia for the patient. Other techniques involve rolling the fat on sterile pads to remove unwanted fluid, or placing the lipoaspirate into a plastic bag with a filter system whereby the bag is manually agitated to separate the fat from the fluid. Yet another method involves removing the bottom layer of fluid from a plastic container that has been modified to include a port or a spigot at the bottom, and then transferring the remining fat to syringes for transfer into the patient. All of these methods require multiple steps that take time, can be tedious, and risk exposure to contamination.

With respect to preparation of harvested adipose tissue, the fat collection canister 10 and system 1 of the present disclosure is uniquely configured to intermittently oxygenate the collected fat once it has been removed from the patient. Heretofore, no such integrated system existed for autologous fat transfer. This aspect represents a material advance in the field, as the introduction of oxygen into the lipoaspirate increases the concentration of dissolved oxygen per unit volume (mg/L), thereby supporting physiologic fat cell viability. From the moment adipose cells are removed from the body and collected within the canister, the cells are deprived of their native vascular supply and corresponding metabolic support. Nor have they yet been reintroduced into the body to rely upon the physiologic mechanism of imbibition during the interim period leading to neovascularization. By increasing the partial pressure of oxygen in solution, the deleterious cascade of hypoxia, anaerobic metabolism, lactic acidosis, and eventual adipocyte death may be mitigated to a clinically meaningful degree.

In cases where autologous fat transfer is desired, the following steps are performed and facilitated by the system 1 herein: intermittent oxygenation of harvested fat by means of a diffuser 54, washing of the fat with antibiotic-infused saline solution, separation of the fat component from the combined lipoaspirate and wash solution, and concentration of the purified fat fraction for reinjection. Conventional fat preparation methods including gravity sedimentation, centrifugation, mesh or colander filtration, Telfa pad rolling, evacuation port technique, and pouch preparation. Each of these methods suffers notable limitations, such as lengthy processing times, restricted throughput that prevents use in large-volume transfers, open and exposed pathways that elevate contamination risks, and small batch syringe-based handling that increases likelihood of fat necrosis, oil cyst formation, or inadvertent intravascular injection.

In contrast, the present system 1 comprises an integrated, closed-system approach to fat preparation that overcomes these deficiencies. The combined apparatus ensures that fat is harvested from the patient, oxygenated, washed, separated, and concentrated within a sealed and sterile environment, and then reintroduced into the patient via the same closed system pathway, reversed for reinfusion. This closed-loop functionality prevents microbial contamination, reduces infection risk, increases fat viability, and streamlines the autologous transfer procedure.

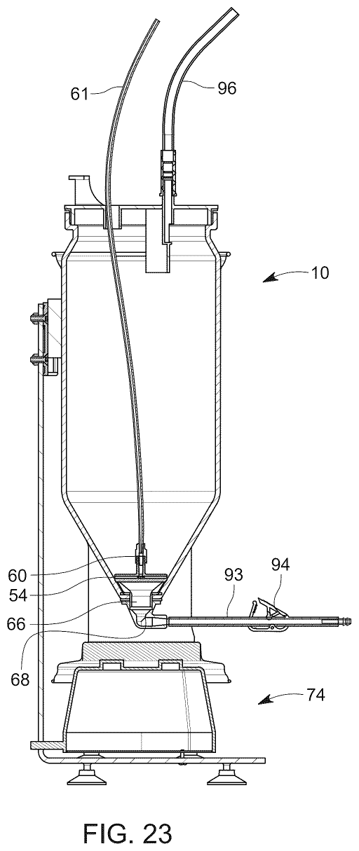

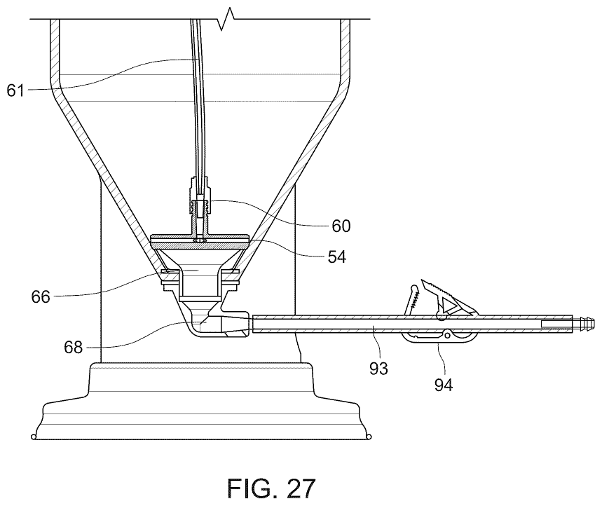

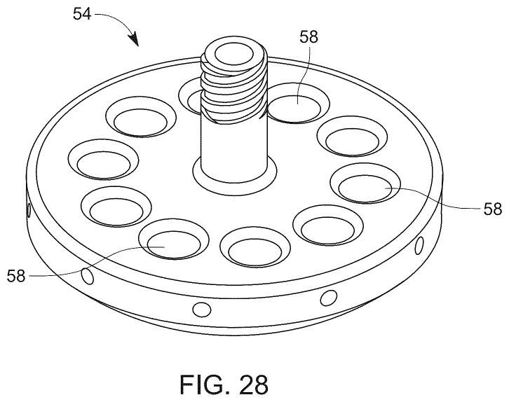

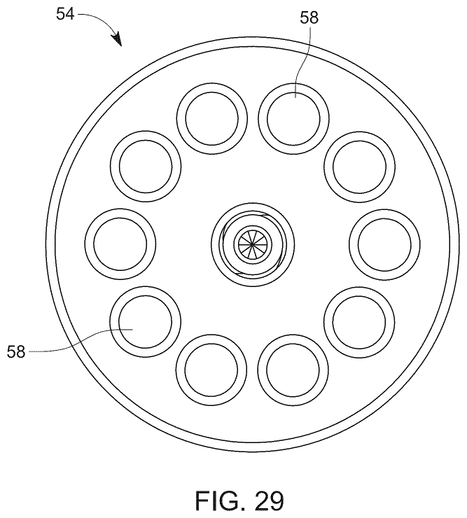

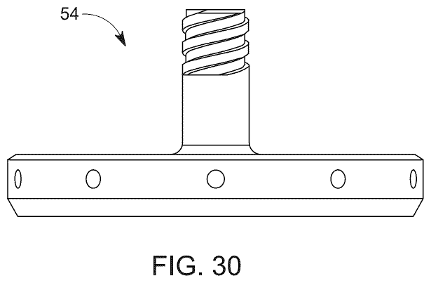

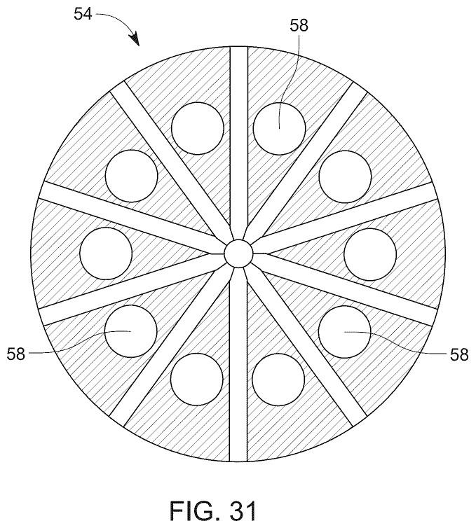

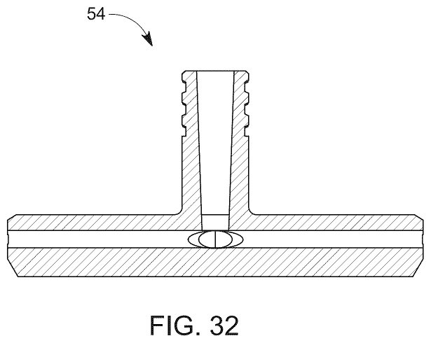

As shown in FIGS. 23 and 27-32, an aeration splitter 54 is incorporated into the present system. The splitter 54 is introduced into the collection canister 10 through an access port 56 provided in the canister 10 lid. Structurally, the splitter 54 is configured to divide the incoming oxygen flow into ten discrete egress apertures 58, thereby reducing the output velocity at each port 56 to one tenth of the incoming velocity. This configuration enables gentle dispersion of oxygen into the lipoaspirate solution. The aeration splitter 54 is preferably formed from stainless steel or comparable biocompatible metal alloys and is reusable following sterilization. The splitter 54 is fluidly coupled to sterile, single-use PVC tubing 61 via a male-to-female Luer lock connector 60. The proximal end of the PVC tubing 61 is then coupled to the outlet of an oxygen tank regulator by means of a barbed connector. The system may employ a specialized calibration regulator affixed to an E-class oxygen tank, said regulator being configured to deliver oxygen at a fixed flow rate of 0.25 liters per minute. At this fixed flow rate, the oxygen egress through the splitter 54 is capped at approximately 0.42 cc O2/sec, which has been determined to prevent both hyperoxygenation injury and mechanical disruption of the fat. The result is a slow, gentle, micro-bubbling of oxygen through the lipoaspirate mass, rising from the base of the canister 10 and permeating throughout the harvested tissue.

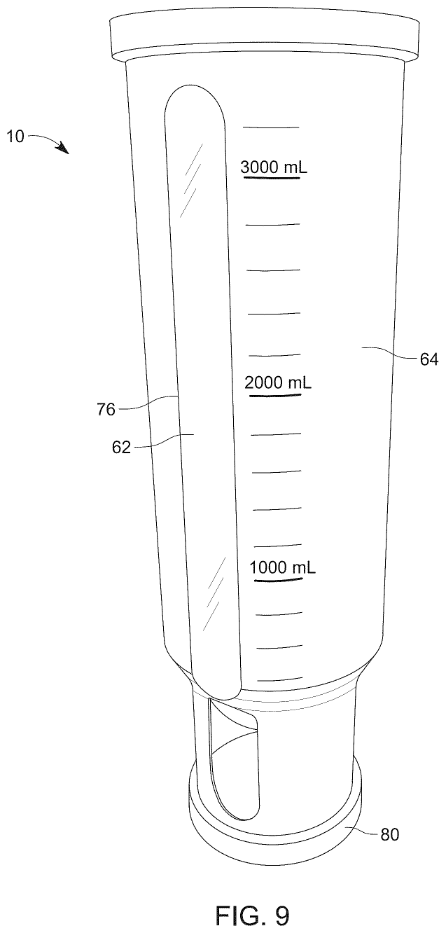

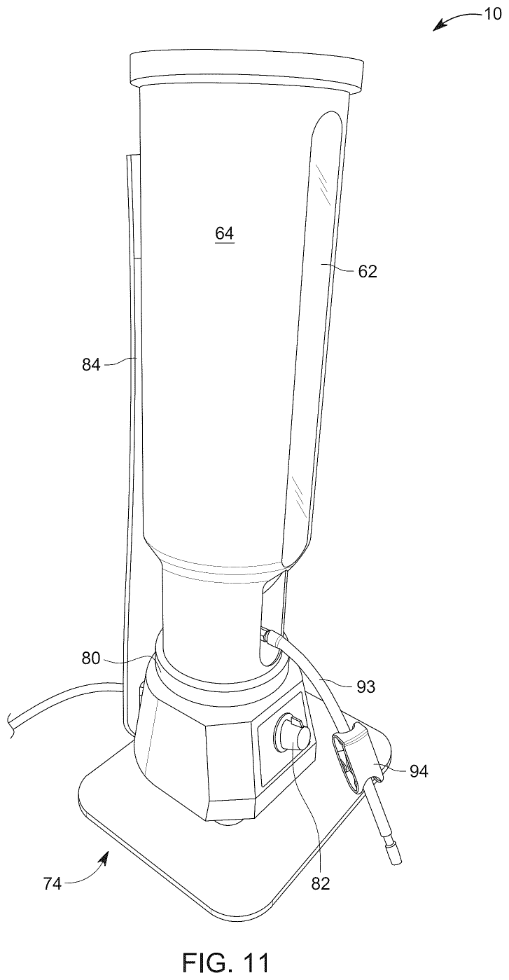

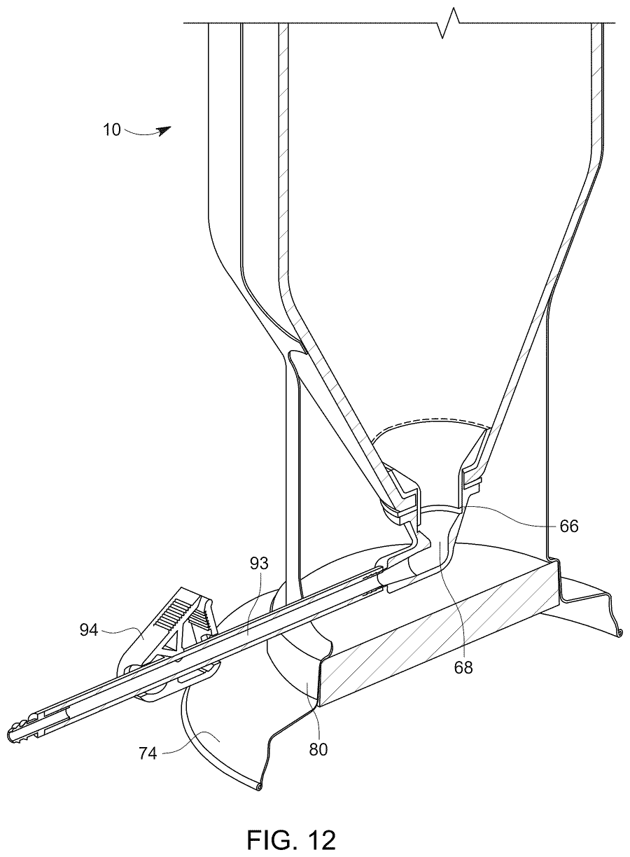

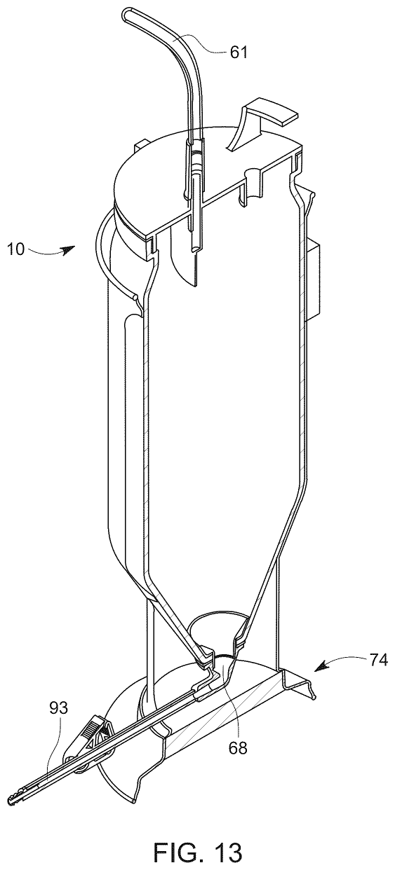

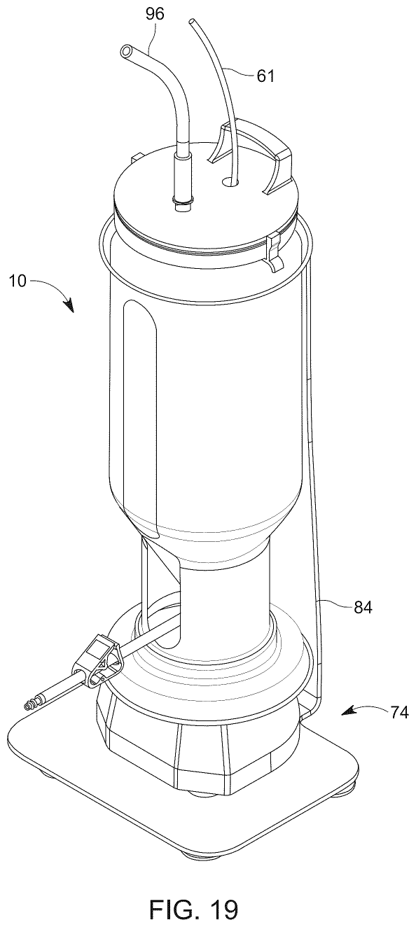

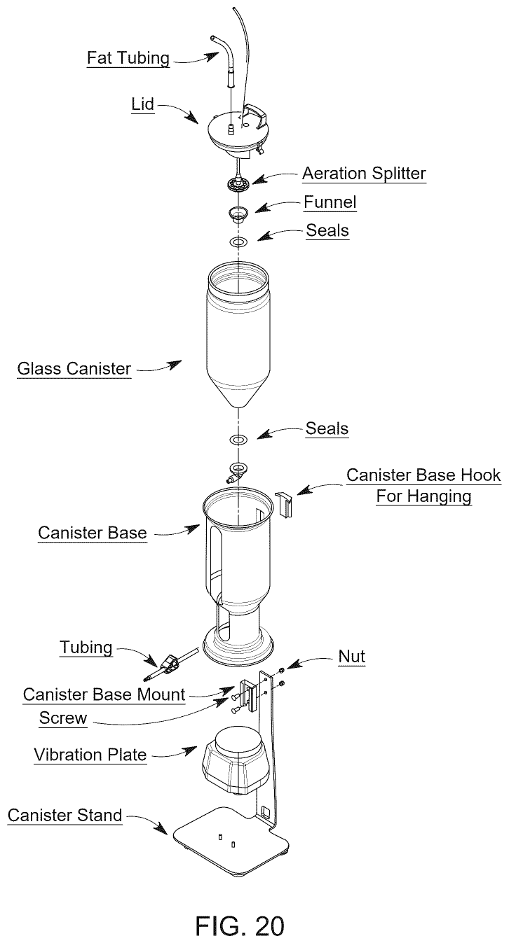

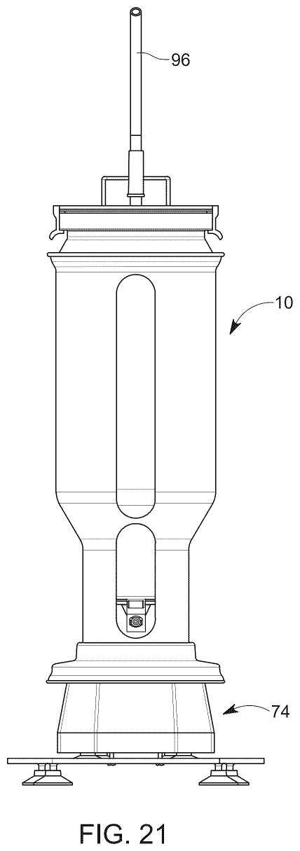

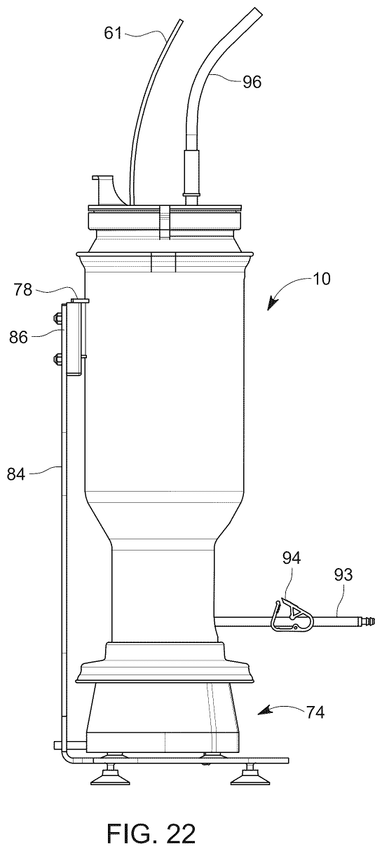

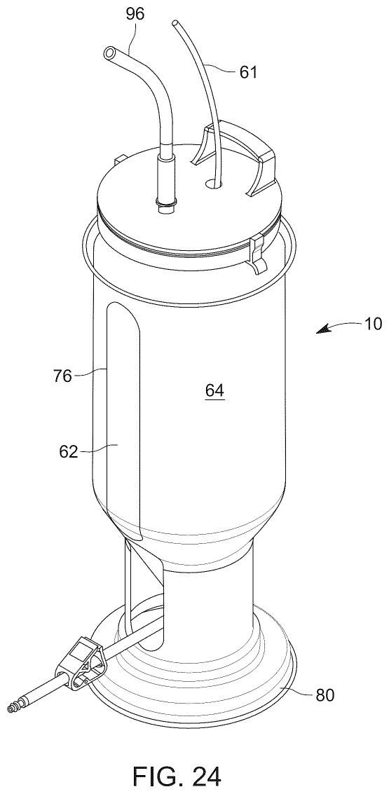

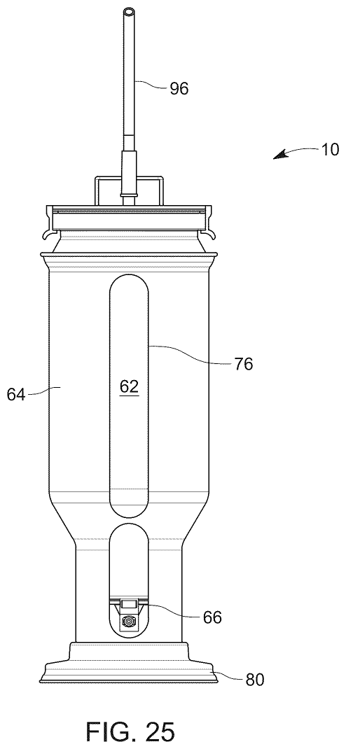

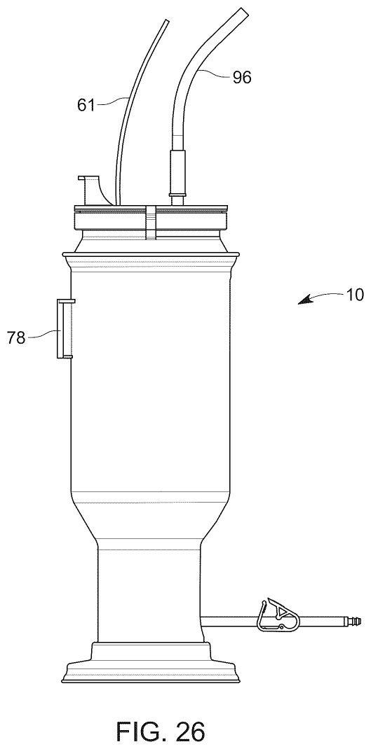

FIGS. 9-11 and 19-27 illustrate exemplary embodiments of a fat collection canister 10 and its operable connections that enable preparation and transfer of harvested fat 88. In one or more embodiments, the collection canister 10 comprises a cylindrical inner container 62 formed of fracture-resistant or tempered glass, encased within an outer protective sleeve 64 constructed of steel or other suitable metal alloy. The sleeve 64 provides mechanical protection, stability, and structural reinforcement to the inner container 62, and may further include a mounting interface 78 such as a hook-and-sleeve assembly configured for coupling the canister 10 to a wall or other support surface. The inner container 62 is tapered at its lower portion and includes an aperture 66 at the bottom center that fluidly communicates with a hollow elbow fitting 68 (FIG. 12) affixed thereto. In one or more embodiments, a Luer lock or equivalent connection mechanism is employed to secure the elbow fitting 68 to the canister bottom.

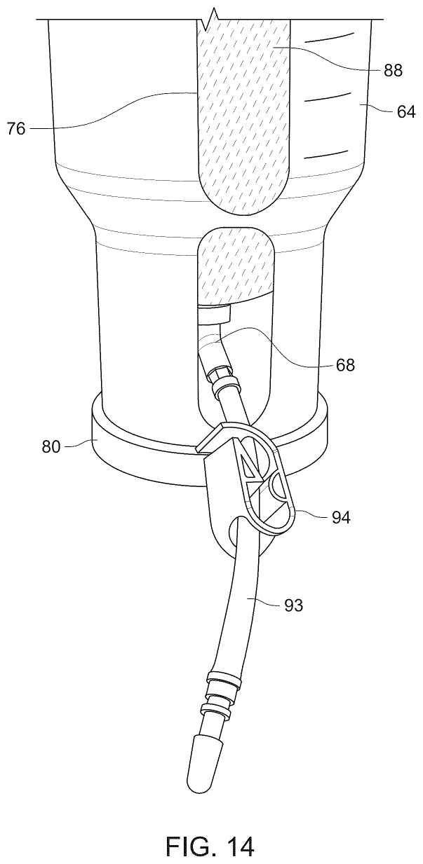

The elbow fitting 68 is specifically configured to prevent clogging by fat tissue during egress from the canister 10 and into downstream tubing. In one embodiment, the proximal portion of the elbow fitting 68 affixed to the canister 10 defines an inner diameter of approximately 8 mm, sufficient to allow free passage of fat tissue without obstruction. The elbow fitting 68 tapers distally to a diameter of approximately 5 mm, whereby the reduced diameter is compatible with the tubing to which the distal end is operably connected. A short connector tube 93, as shown in FIG. 14, may be removably attached to the distal end of the elbow fitting 68. This connector tube 93 is adapted for selective attachment either to oxygenation tubing 61 during intermittent oxygenation of lipoaspirate or to fat transfer tubing 96 during autologous fat transfer. The connector tube 93 is further equipped with a clamp 94 for sealing the tube 93 during suction and separation modes, thereby maintaining sterility and containment of lipoaspirate, and for release during fat transfer to allow drainage and forward pumping of the fat.

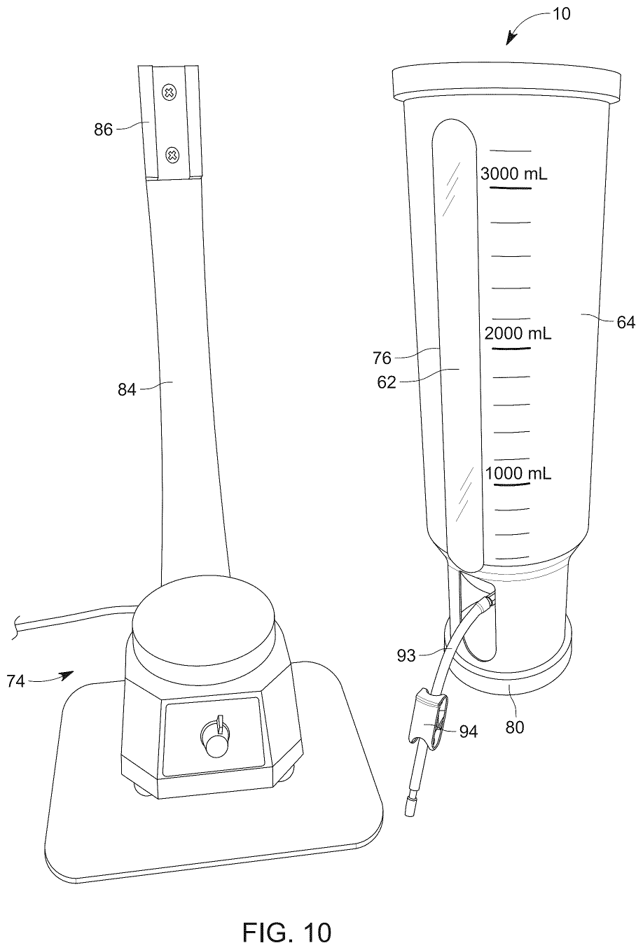

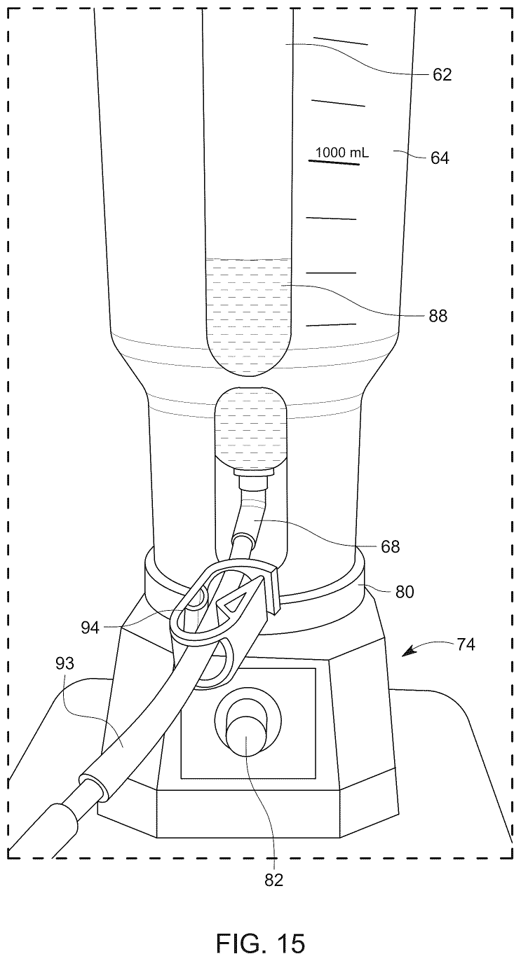

In a preferred embodiment, the encasing sleeve 64 not only protects the inner container 62 from breakage but also serves as an interface for operable connection with a vibrational plate 74 used in fat separation. As illustrated in FIGS. 9 and 10, the sleeve 64 includes a vertically oriented cutout window 76 that extends substantially the height of the canister 10, permitting direct visualization of lipoaspirate separation and fluid levels, while also allowing access to the elbow fitting 68 during downstream transfer. The sleeve 64 further includes a slide connector 78 disposed on the rear side for removable engagement with a corresponding connector 86 disposed on a suction machine and on the vibrational plate 74. The lower section of the sleeve 64 is tapered to match the container 62 and incorporates a circumferential lip 80 configured to seat securely onto the round surface plate of the vibrational platform 74 (FIG. 15).

Upon completion of the liposuction phase, the collection canister 10 is detached from vacuum and other tubing connections, such as the suction pathway from the quad tubing 36 assembly, and is mounted upon the vibrational plate 74 via the slide connector 78 and base lip 80 interface. The vibrational plate 74 comprises an internal motor, adjustable via a rotary dial 82, configured to impart vibrational energy to the canister 10. The vibrational plate 74 further includes a vertical stabilization arm 84 with an attachment point 86 that mates with the slide connector 78 of the canister sleeve 64. Once the canister 10 is engaged and secured, an antibiotic saline wash may be introduced into the canister 10 to rinse the harvested tissue. The vibrational plate 74 is then actuated, with vibration magnitude and duration adjusted to promote separation of purified fat from tumescent and wash fluids. Tissue variability, including fibrous content, density, and fat-to-fluid ratio, determines the exact vibrational profile, which may be selected by the attending surgeon or operative personnel. During this separation phase, oxygenation of the tissue may optionally be applied in accordance with the methods described above.

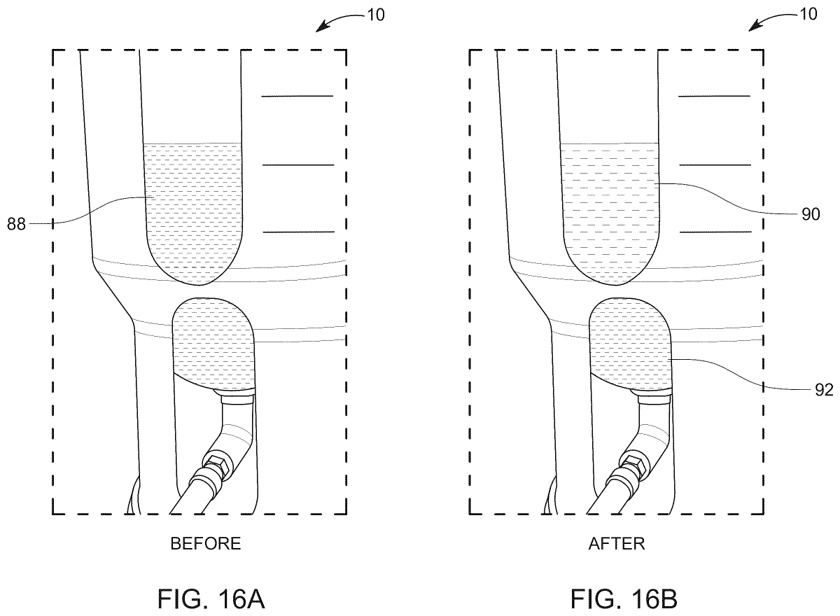

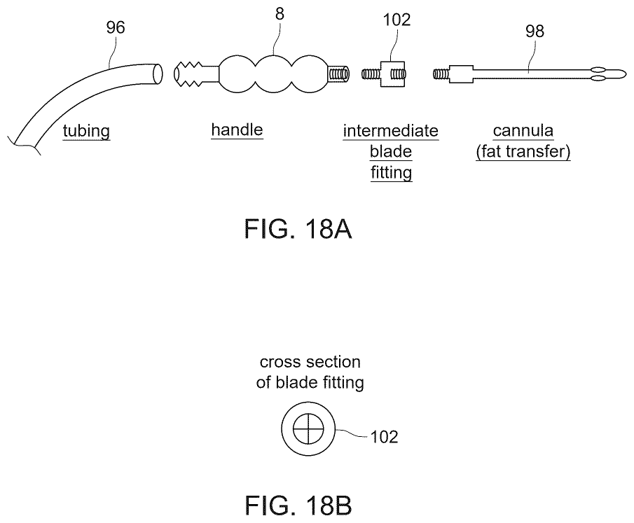

As illustrated in FIGS. 16A and 16B, separation results in concentration of purified fat 88 into an upper buoyant layer 90 above the residual fluid layer 92, which may be visually monitored through the cutout window 76 of the sleeve 64. Once adequate separation (on the order of approximately 1-10 minutes) is reached, the clamp 94 on the connector tube 93 may be released to allow drainage of excess fluids, leaving primarily the separated fat for transfer. At this stage, the proximal end of a fat transfer tubing 96 segment is operably coupled to the attachment point of the connector tube 93, while the distal end is secured to a fat transfer cannula 98 via an ergonomic handle interface (FIG. 18A). The fat transfer tubing 96 is routed through a peristaltic pump 4, which is selectively actuated by surgeon depression of the corresponding pedal 15 on the wireless triple pedal 14. The activated peristaltic pump 4 draws fat 88 from the canister 10, through the fat transfer tubing 96, and delivers it into the fat transfer cannula 98 for reinjection into a target patient site.

Fat transfer cannulas 98 generally contain small egress apertures at a distal tip through which fat exits into the target site. Such apertures are susceptible to clogging, particularly when aspirated material contains fibrous fat tissue. To mitigate this, the present invention incorporates an intermediate threaded fitting 102 disposed between the ergonomic handpiece 8 handle and the fat transfer cannula 98. As illustrated in FIG. 18B, this threaded fitting 102 comprises cutting means such as blades, ridges, or barbs disposed along its interior circumference. In use, adipose tissue is fragmented into smaller sections as it passes through the fitting 102, thereby reducing clogging at the cannula apertures and supporting continuous, uniform fat transfer.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Spatially relative terms, such as “below”, “beneath”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. Throughout the specification, like reference numerals in the drawings denote like elements.

Embodiments of the inventive subject matter are described herein with reference to plan and perspective illustrations that are schematic illustrations of idealized embodiments of the inventive subject matter. As such, variations from the shapes of the illustrations as a result, for example, of manufacturing techniques and/or tolerances, are to be expected. Thus, the inventive subject matter should not be construed as limited to the particular shapes of objects illustrated herein, but should include deviations in shapes that result, for example, from manufacturing. Thus, the objects illustrated in the figures are schematic in nature and their shapes are not intended to illustrate the actual shape of a region of a device and are not intended to limit the scope of the inventive subject matter.

Unless otherwise defined, all terms (including technical and scientific terms) used herein have the same meaning as commonly understood by one of ordinary skill in the art to which this present inventive subject matter belongs. It will be further understood that terms used herein should be interpreted as having a meaning that is consistent with their meaning in the context of this specification and the relevant art and will not be interpreted in an idealized or overly formal sense unless expressly so defined herein. The term “plurality” is used herein to refer to two or more of the referenced item. Although any methods, devices, and materials similar or equivalent to those described herein can be used in the practice or testing of the presently disclosed subject matter, representative methods, devices, and materials are now described.

In the drawings and specification, there have been disclosed typical preferred embodiments of the inventive subject matter and, although specific terms are employed, they are used in a generic and descriptive sense only and not for purposes of limitation, the scope of the inventive subject matter being set forth in the following claims.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A liposuction and autologous fat transfer system comprising:
a regulator box comprising a wireless receiver and a central processing unit;
a peristaltic pump operably coupled to the regulator box for controllable delivery of tumescent solution and fat transfer;
a suction machine operably coupled to a collection canister;
a collection canister comprising an inner cylindrical container fitted within an outer sleeve;
a pedal unit comprising a plurality of pedals, wherein each pedal is configured to independently activate or deactivate one or more of the regulator box, the peristaltic pump, or the suction machine;
a handpiece comprising a cannula and operably connected to the peristaltic pump and the suction machine; and
a vibrational plate comprising;
a base platform configured to receive and support the collection canister;
a motor disposed within the base platform, operable to impart vibrational energy to the supported collection canister; and
wherein the vibrational energy facilitates separation of fat tissue within the collection canister into distinct layers.

2. The liposuction and autologous fat transfer system of claim 1, further comprising a disposable, bonded tubing assembly having a plurality of separable tubes configured for fluid connection to the regulator box, the peristaltic pump, the collection canister and the liposuction handpiece.

3. The liposuction and autologous fat transfer system of claim 1, further comprising:
an aeration splitter configured for introduction through an access port in the collection canister, the aeration splitter comprising a plurality of egress apertures for dividing an incoming oxygen flow and effecting micro-bubbling dispersion of oxygen throughout lipoaspirate contained in the canister.

4. The liposuction and autologous fat transfer system of claim 1, wherein the regulator box further comprises:
a front user interface including a rotary dial for manual adjustment of air pressure output and a digital display showing the real-time output pressure;
a transparent door panel covering an accessible oil filter to facilitate visual inspection and maintenance without internal disassembly;
rear-panel accessibility to at least the oil filter, wireless receiver, and muffler, the oil filter positioned for removal and replacement without exposing internal electrical components;
and a multi-channel relay actuated by either wireless or pneumatic pedal input signals and controlled by the central processing unit contained on a custom printed circuit board therein.

5. The liposuction and autologous fat transfer system of claim 2, wherein the bonded tubing assembly further comprises:
at least four discrete, separable tubes for simultaneously providing air-in, air-out, tumescent fluid, and suction connections between the handpiece and system components;
each tube further including externally ultrasound-welded, custom-molded fittings dimensioned to maintain a substantially uniform inner diameter at every connection point, wherein the fittings are configured for single-use disposability; and
wherein the fittings are engineered to withstand elevated pneumatic pressures without detachment or loss of seal.

6. The liposuction and autologous fat transfer system of claim 5, wherein each tube has an inner diameter of at least 5 mm and being formed of single-lumen PVC longitudinally bonded together to permit peeling at desired locations.

7. The liposuction and autologous fat transfer system of claim 1, wherein the pedal unit is a wireless triple pedal unit comprising at least three pedals and a wireless transmitter, wherein each pedal is configured to independently activate or deactivate one or more of the regulator box, the peristaltic pump, or the suction machine via communication with the wireless receiver.

8. The liposuction and autologous fat transfer system of claim 1, wherein:
a bottom of the inner cylindrical container includes an aperture in fluid communication with a hollow elbow fitting configured to minimize clogging by adipose tissue such that contents in the container can be drained from the container through the aperture and out of the elbow fitting;
a bottom of the outer sleeve includes a lip having a circumference generally corresponding to a circumference of the base platform of the vibrational plate;
the outer sleeve has a vertically oriented cutout portion that allows visibility of the inner cylindrical container; and
the collection canister can be affixed to a vertical arm of the vibrational plate.

9. The liposuction and autologous fat transfer system of claim 1, wherein the vibrational plate further includes;
a user-adjustable control configured to vary at least one parameter selected from the group consisting of vibration frequency, amplitude, and duration; and
a vertical arm extending upward from a rear position of the base platform to secure the collection canister in position during vibration.

10. The liposuction and autologous fat transfer system of claim 1, further including a threaded fitting disposed between the handpiece and the cannula, whereby the threaded fitting comprising cutting means along an interior circumference configured for fragmenting adipose tissue into smaller segments.

11. A method of performing liposuction and autologous fat transfer comprising the steps of:
providing the system of claim 1;
selectively vibrating the cannula during infiltration and aspiration using air pressure regulated by the regulator box;
controlling tumescent solution delivery via the peristaltic pump actuated by the wireless triple pedal unit;
collecting lipoaspirate in the collection canister;
oxygenating collected fat using the aeration splitter;
separating fat and fluid in the canister by subjecting the canister to vibrational energy with the vibrational plate; and
transferring purified fat via a closed-loop pathway to a fat transfer cannula using the peristaltic pump and dedicated fat transfer tubing.

12. The method of claim 11, further comprising:
placing the collection canister onto a vibrational plate after completion of suction, said vibrational plate operatively connected via a slide connector and stabilization arm;
introducing an antibiotic saline wash into the canister and activating the vibrational plate to impart mechanical energy to the canister for a selectable time and frequency, thereby promoting separation of a purified fat layer from residual fluid; and
visually monitoring the separation via an elongated cutout window in a protective sleeve surrounding the canister, and draining the lower fluid layer through a removable connector tube upon achieving desired separation.

13. A method of performing liposuction and autologous fat transfer comprising the steps of:
providing the liposuction and autologous fat transfer system of claim 1;
providing vibration to the liposuction handpiece;
injecting tumescent fluid into the patient;
providing suction to the liposuction handpiece;
suctioning lipoaspirate from the patient into the collection canister;
removing the tubing from the collection canister;
placing the canister on the vibrational plate;
vibrating the lipoaspirate until sufficient separation of fat from fluid is achieved;
draining the fluid from the collection canister;
attaching a proximal end of a suction tube to the elbow fitting of the canister;
attaching a distal end of the suction tube to a fat transfer cannula;
running the suction tube through the peristaltic pump; and
injecting fat from the collection canister into the patient.

14. A method of performing a liposuction procedure, comprising the steps of:
delivering infiltrating fluid to a target tissue site via a fluid delivery device;
selectively actuating a vibrational energy source operatively coupled to a cannula positioned within the target tissue site;
aspirating adipose tissue through the cannula into a collection canister;
intermittently oxygenating the aspirated adipose tissue within the collection canister;
separating the adipose tissue from residual fluids within the collection container using vibrational energy;
transferring the separated adipose tissue from the collection container to a delivery cannula; and
reinjecting the separated adipose tissue into a patient.

15. The method of claim 14, wherein:
the collection canister comprises an inner cylindrical container and an outer sleeve disposed about the inner cylindrical container;
wherein the outer sleeve is configured to be secured upon a vibrational plate that provides the vibrational energy for separating the adipose tissue from the residual fluids; and
wherein the inner cylindrical container comprises an aeration splitter for oxygenating the adipose tissue.

16. The method of claim 14, further including the step of providing a disposable, bonded tubing assembly having a plurality of separable tubes configured for fluid connection to at least the fluid delivery device, the collection canister, and the cannula.

17. The method of claim 14, further including the step of providing a regulator box comprising a central processing unit and a wireless receiver.

18. The method of claim 14, further including the step of providing a wireless triple pedal unit comprising at least three pedals and a wireless transmitter, wherein each pedal is configured to independently activate or deactivate one or more components configured to execute the steps of the method.

19. A method of liposuction and autologous fat transfer comprising the steps of:
infiltrating a tumescent solution into a target tissue region of a patient by actuating a vibrating cannula, said vibration generated by an air-powered handpiece in fluid communication with a regulator box controlled wirelessly;
aspirating lipoaspirate from the patient using suction applied to the vibrating cannula, the suction being provided by a suction machine connected via the handpiece and a collection canister, wherein all device activation is wirelessly controlled;
collecting the lipoaspirate in the fat collection canister, said canister comprising a cylindrical inner container and an outer protective sleeve with a cutout window;
intermittently introducing oxygen into the collected lipoaspirate using an aeration splitter having multiple egress apertures to provide low-velocity micro-bubbling;
mounting the canister onto a vibrational plate and actuating the vibrational plate to promote separation of purified fat into an upper layer above residual fluid;
draining excess fluid from the canister via a drainable connector tube, thereby isolating the purified fat; and
transferring the purified, oxygenated fat from the canister to a fat transfer cannula using a peristaltic pump and dedicated transfer tubing, thereby enabling reinjection of the fat into a patient site via a closed-system pathway.
